(19) United States
(12) Patent Application Publication
(10) Pub. No.: US 2015/0190095 A1
(43) Pub. Date: Jul. 9, 2015

(54) MEDICAL IMAGING APPARATUS TO DETECT A MOVING PART OF A PATIENT'S BODY

(71) Applicant: ISP SYSTEM, Vic-en-Bigorre (FR)

(72) Inventors: Paul Sauvageot, Vic-en-Bigorre (FR); David Brescon, Vic-en-Bigorre (FR); Jean-Marc Cazalet, Vic-en-Bigorre (FR)

(21) Appl. No.: 14/412,776

(22) PCT Filed: Jun. 26, 2013
(86) PCT No.: PCT/FR2013/000163

§ 371 Date: Jan. 5, 2015

(30) Foreign Application Priority Data
Jul. 5, 2012 (FR) 12/01900

(51) Int. Cl. A61B 5/00; A61B 6/03; A61B 5/055; G06T 7/00; G06T 7/20

(52) U.S. Cl. CPC A61B 5/721; G06T 7/0016; G06T 7/0038; G06T 7/2006; A61B 5/055; A61B 6/03; A61B 6/037; G06T 2207/30061; G06T 2207/30196; G06T 2207/10072; G06T 2207/10081; G06T 2207/10088; A61B 2560/0228

(57) ABSTRACT

A medical imaging apparatus and a device to detect the position of a moving part of the body of a patient. The detection device includes a management module including electronic and electric components including a differential pressure sensor linked to a measuring device that generates a differential pressure representative of the respiration flow rate of the patient, the management module being organized so as to perform at least: one acquisition and processing of the data produced by the differential pressure sensor for the generation of data representative of the respiratory volume and for the generation of an outgoing digital synchronization signal; the reception of an incoming digital synchronization signal; the generation of an outgoing digital signal of data representative of the respiratory volume in line with one of the two synchronization signals.

The technical scope of the present invention is that of apparatus used for medical purposes. The present invention namely relates to devices to detect the position of a moving part of a patient's body analysed by medical imaging.

Medical imaging devices are apparatus subjected to stringent standards which enable complex data to be obtained using elaborate processes such as positron emission tomography (PET), computed tomography acquisition (CT) or magnetic resonance imaging (MRI). During the acquisition of medical images, the patient's health must be protected and any interaction likely to perturb the medical imaging devices and increase the risk of accident as such images are taken prevented.

For safety reasons, a CT imaging device which emits X-rays to the patient is not able to receive a communication signal from another apparatus. According to current applicable standards, no input for receiving data is allowed in a CT imaging device namely to avoid their causing errors in the X-ray emission control. A CT imaging device generally only comprises a single output supplying an outgoing digital synchronization signal.

For safety reasons, a PET imaging device generally only comprises one input of a digital synchronization signal, no other data exchange being permitted according to current standards.

The complex imaging data supplied by the different imaging devices may be combined and processed so as to extract new information.

A PET examination, for example, enables the position of high activity cells to be detected with the aid of a radioactive marker. Cancerous cells are namely detected in this way. A CT scan enables the PET examination to be completed by supplying the position of the patient's organs. Thus, the compilations of the two types of information enable the cancerous cells to be located in the patient's organs.

One problem arises, however, in the case of medical imaging applied to moving organs, such as, for example, the lungs. A patient is not able to remain immobile and to stop breathing during an examination which may last for more than ten minutes. It is necessary, in that case, to detect the positions of the patient's body and to compile the data supplied by the imaging devices in correlation with the data representative of the position of the patient's body. The data representative of the position of the patient's body is generally supplied by a device to visualize the patient's diaphragm which comprises a camera in whose field a marker is positioned, attached to the diaphragm. Each position of the patient may then be determined by image analysis.

However, analyses performed with this type of device to detect the position of the patient's diaphragm are generally lacking in accuracy. The failure rate depending on the patient is of 30% or even 66% according to the different clinical studies performed. It is, in fact, difficult to determine the positions of moving organs in a three-dimensional space based on a movement detected in two dimensions. The slightest measuring error generally causes the data representative of the position of the organs to be invalidated.

Another problem lies in that the imaging devices used with a device to visualize the patient's diaphragm requires a data acquisition that is approximately 20% longer. In the case of an X-ray imaging device, the patient is exposed to X-rays at a 20% higher rate. Therefore, for each patient, the chances of successfully achieving the data acquisition for the medical imaging must be evaluated in view of the expected benefit. If successful, this enables the more accurate detection, on the one hand, of any tumour activity and, on the other, of the pathological volumes.

There thus appears a need to enhance the reliability of the detection of the position of a moving body for physiological measuring apparatus and namely for medical imaging devices.

The aim of the present invention is to overcome several of the drawbacks to prior art by supplying a new device to detect the position of a moving part of a patient's body analysed by medical imaging.

The invention thus relates to a device to detect the position of at least one moving part of a patient's body analysed by medical imaging, wherein it comprises a management module comprising electronic and electric components including at least one differential pressure sensor linked to a measuring device generating a representative differential pressure of the patient's respiratory flow rate, the management module being organised to perform at least:
the acquisition and processing of data produced by the differential pressure sensor for the generation of data representative of the respiratory volume and for the generation of an outgoing digital synchronization signal,
the reception of an incoming digital synchronization signal,
the generation of an outgoing digital signal of data representative of the respiratory volume in line with one of the two incoming or outgoing digital synchronization signals.

According to one characteristic of the invention, the outgoing digital synchronization signal is representative of the detection of extremes with respect to the respiratory movement corresponding to the minimum or maximum respiration flow rate of a defined portion of the respiratory cycle.

According to another characteristic of the invention, the management module is adapted for the reception of a selection command for one of the two incoming or outgoing digital synchronization signals for the generation of the outgoing digital signal of the data representative of the respiratory volume in line with one of the two incoming or outgoing digital synchronization signals.

According to another characteristic of the invention, the management module is organized so as to generate the digital signal of the data representative of the respiratory volume in line with the incoming or outgoing digital synchronization signal, with a response time of less than or equal to 30 ms or 15 ms or even 12 ms with respect to the variation in data produced by the differential pressure sensor.

According to another characteristic of the invention, the data representative of the respiratory volume is calculated from a memorized parameterizable model corresponding to a curve representing the volume of air exhaled and inhaled by a patient, the data representative of the patient's respiratory flow rate being processed by a parameterizing module of the model over a pre-determined number of respiratory cycles for a memorization of data representative of the parameterized model, then being processed by a real-time adjustment module of the parameterized model for the generation of respiratory volume data representative.

According to another characteristic of the invention, the detection device comprises a portable case enclosing at least the management module and offset with respect to the moving part being analysed.

According to another characteristic of the invention, the detection device comprises an inhalator through which the patient breathes, this inhalator being linked to a tube connected at the inlet of the measuring device, the measuring device being attached to the case.

Another subject of the invention relates to medical imaging apparatus comprising at least one medical imaging device synchronized with a device to detect the position of the moving part of the patient's body being analysed by medical imaging according to the invention.

According to another characteristic of the invention, the medical imaging apparatus comprises at least two medical imaging devices each of a different type and being of the type positron emission tomography (PET), computed tomography acquisition (CT) or magnetic resonance imaging (MRI).

According to another characteristic of the invention, the medical imaging apparatus comprises a calibration tool for the detection device and said medical imaging devices, the calibration tool comprising an aperture supplied by a chamber and connected to the measuring device, the chamber being delimited by a mobile wall linked to a target able to be detected by said medical imaging devices, the target and mobile wall being joined for the simultaneous control of their displacement, the calibration tool ensuring a synchronization of the timers and medical imaging devices.

A first advantage of the invention lies in the fact that the measurement of the respiratory flow in real-time enables a better correlation with the data supplied by the imaging devices thereby being able to reduce the failure rate to approximately 10% or less than 10%.

Another advantage of the invention lies in the fact that the analysis of the data is more accurate and the tumours detected are smaller. Accuracy is considerably improved. This enables therapy developed on the basis of the data supplied by the medical imaging device(s) to be better adapted.

Another advantage lies in the fact that the detection device is adapted to practically all patients whatever their respiratory mode.

Yet another advantage lies in the fact that the real-time detection of the position of a body according to the invention is not subject to the drifting of the respiratory signal detected over time because, namely in the case of a detection device using a camera, of the displacement of a sensor positioned on the patient's body. The real-time detection device according to the invention is thus better correlated to the kinematic movements of the internal organs, thereby optimising the analysis of the data representative of the body's position, namely for the reconstruction algorithms aiming to supply a representation of the patient's body in two or three dimensions.

Other characteristics, advantages and particulars of the invention will become more apparent from the additional description hereafter of the embodiments given by way of illustration and with reference to the drawings, in which:

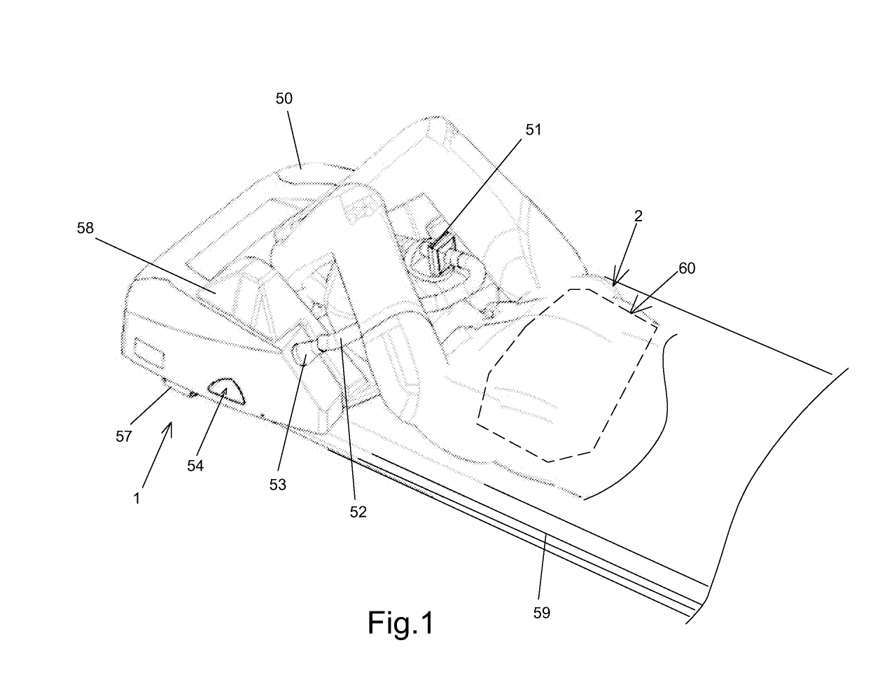

FIG. 1 is a perspective view showing the top of a patient's body and a detection device arranged at one end of a mobile medical table;

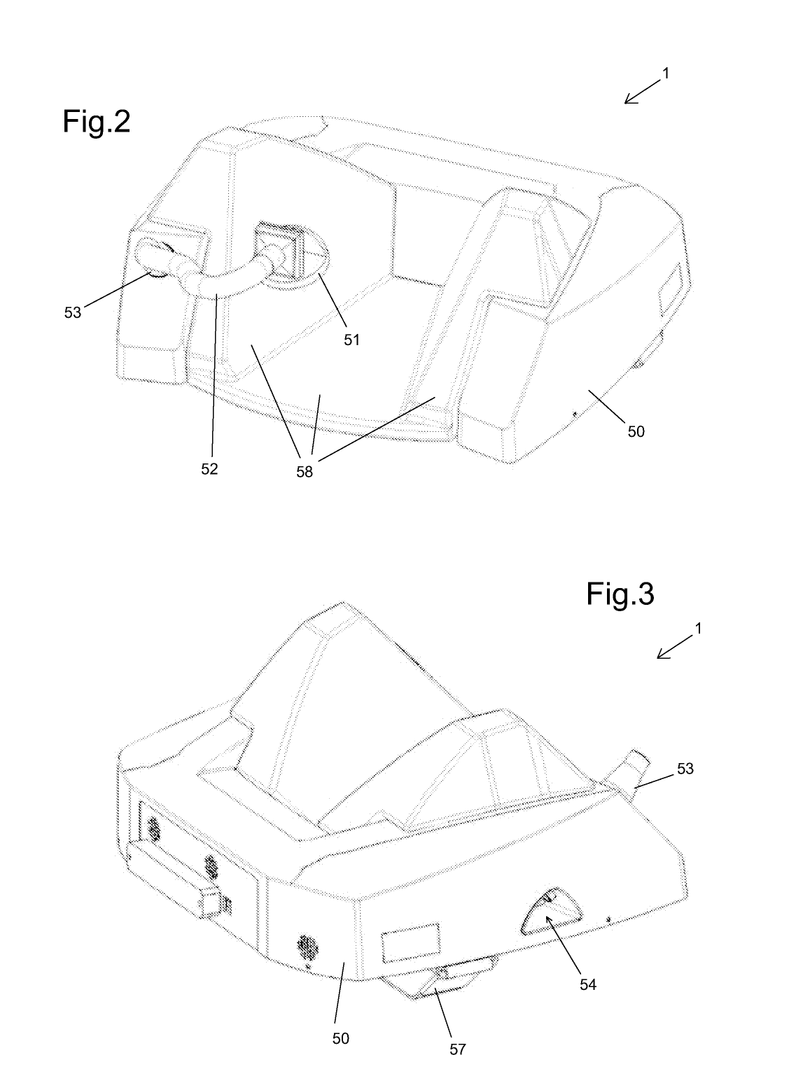

FIG. 2 shows a perspective view of the front of the device to detect the position of a patient's thorax;

FIG. 3 is a perspective view of the rear of the device to detect the position of a patient's thorax;

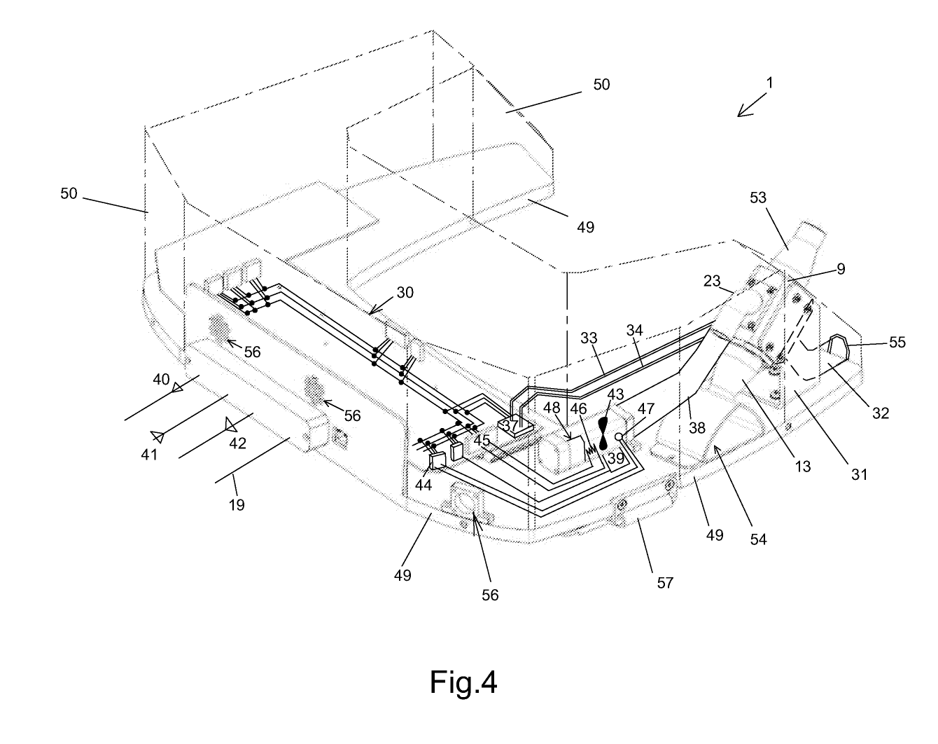

FIG. 4 is a perspective view of the inside of a device to detect the flow rate and volumes in correlation with the position of a patient's thorax;

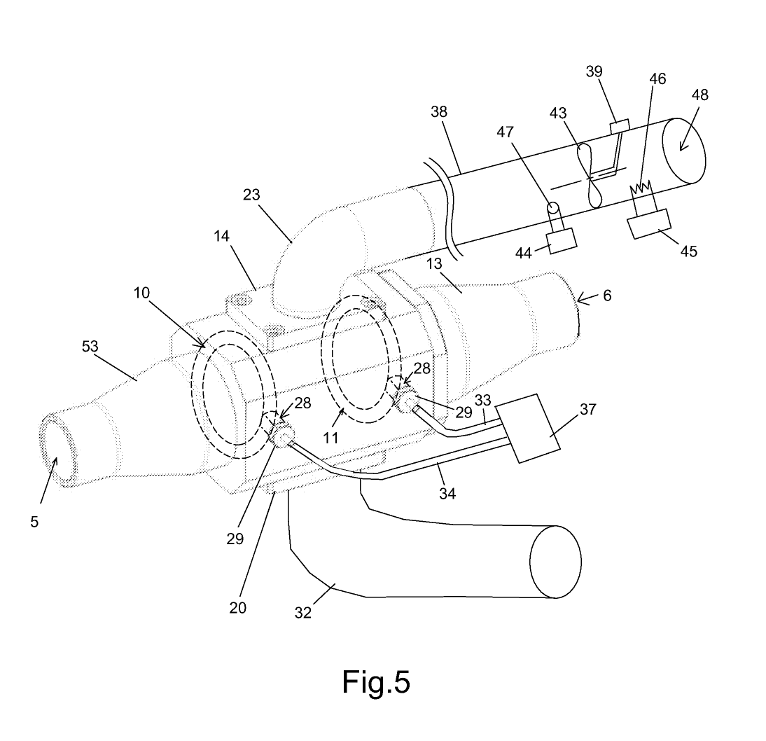

FIG. 5 shows a perspective view of a differential pressure generating device in connection with ducts for the pressure measurements;

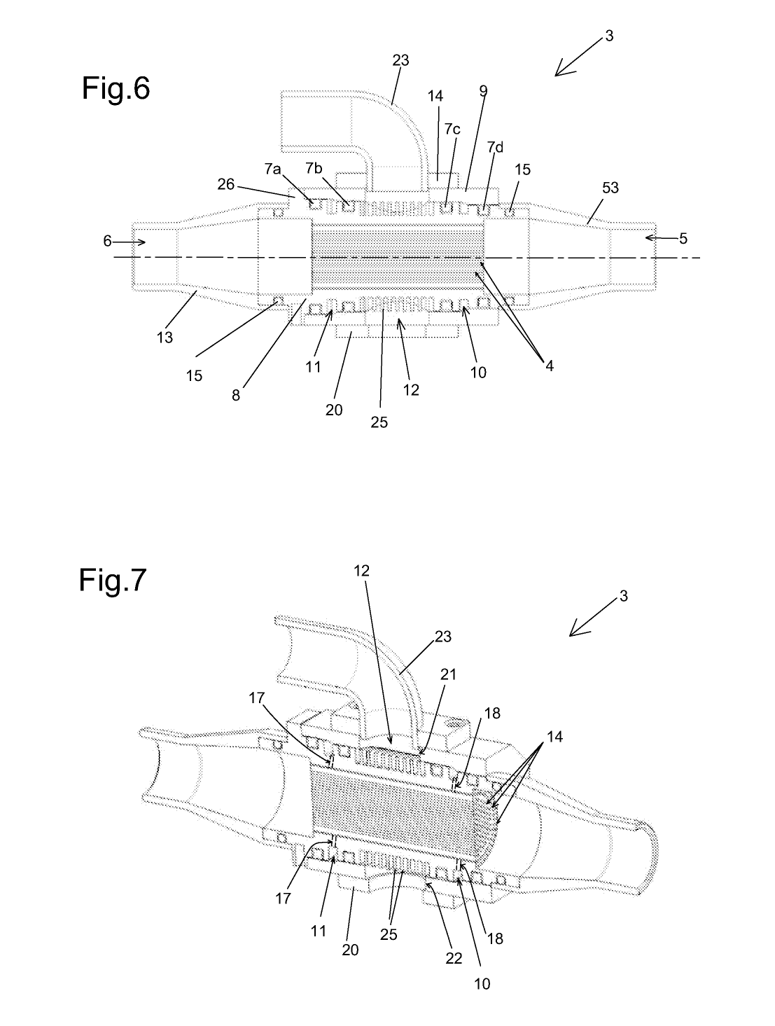

FIG. 6 shows a longitudinal section view of a differential pressure generating device;

FIG. 7 shows a perspective view of the longitudinal section of the differential pressure generating device in FIG. 6;

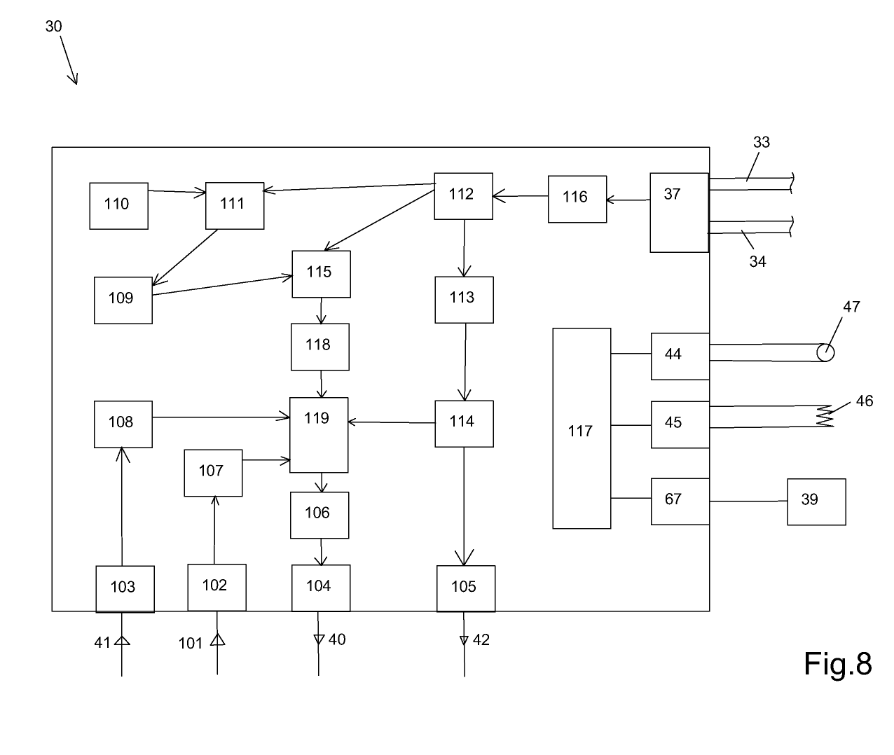

FIG. 8 shows a diagram of a management module for the processing of data and the generation of output signals;

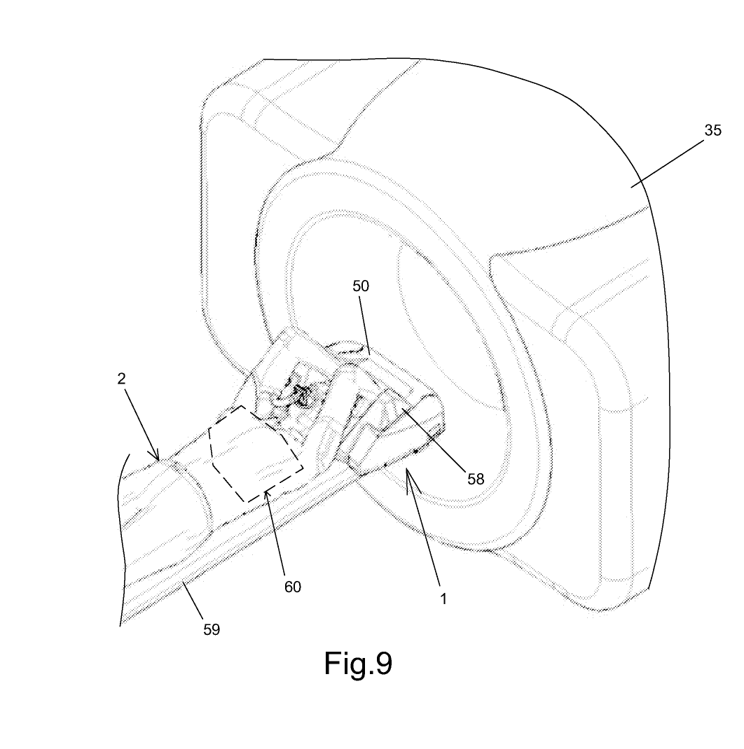

FIG. 9 shows a perspective view of the patient lying on the mobile medical table, with the body position detection device, entering the medical imaging device;

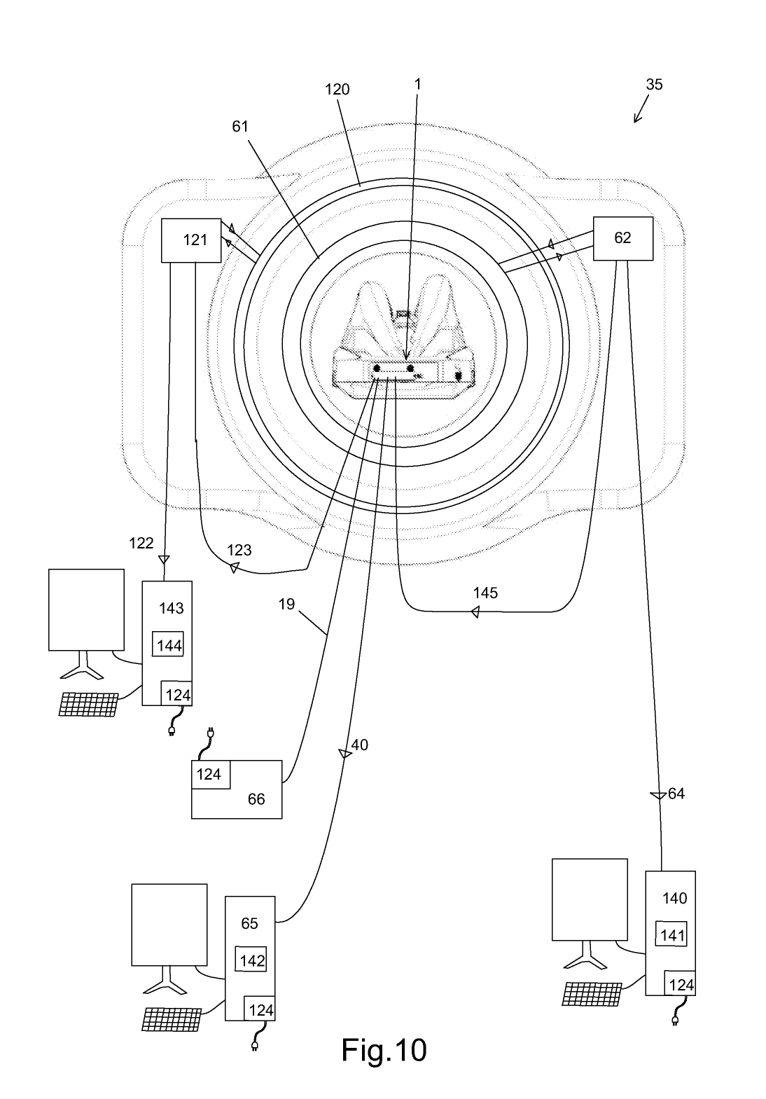

FIG. 10 shows a diagram showing a patient lying on a mobile medical table arranged, with the device to detect the position of the patient's body, in a medical imaging device; and

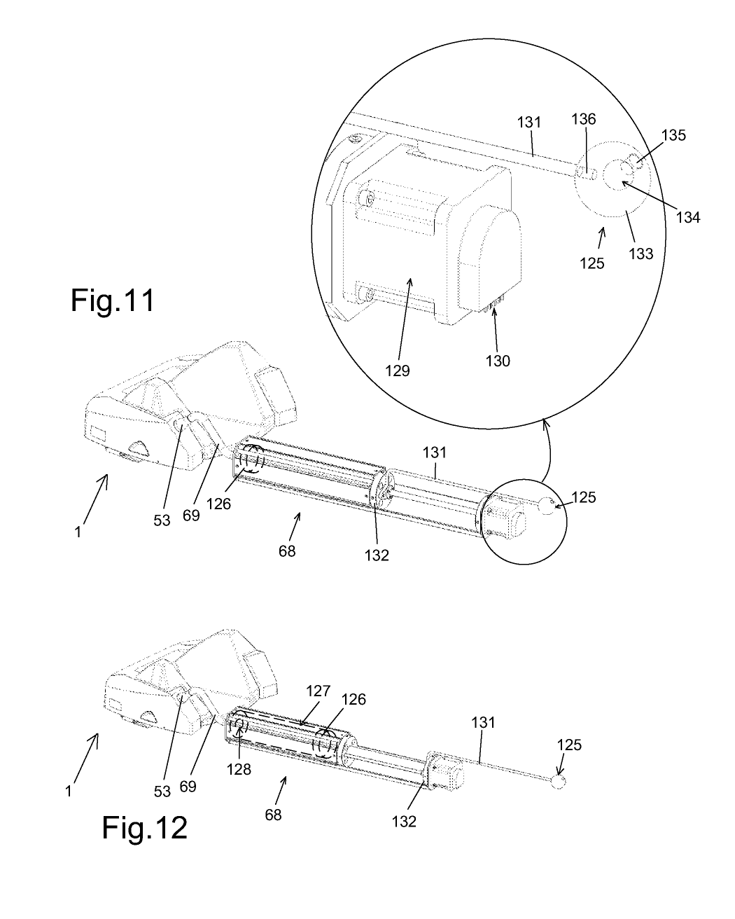

FIGS. 11 and 12 show a calibration device intended to be introduced, with the device to detect the moving part of a patient's body, in a medical imaging device.

The invention will now be described in further detail. In the Figures, the same references are used to designate the same elements.

FIG. 1 shows a patient lying on a table 59. The table 9 is, for example, able to translate horizontally so as to be inserted into medical imaging apparatus. A detection device is attached to the examination table 59 by means of the lower strap 57 of the case.

The patient's head 2 rests on foam wedge cushions 58 and between the branches of a U-shaped shell. Throughout the medical examination, the patient breathes through the inhalator 51, the linking tube 52 and the measuring device opening into a space 54 in the open air.

An area 60 being analysed during the examination is shown on the thorax of the patient 2.

Depending on the type of medical imaging device used, shielding may be provided for all the electronic parts. A shielded shell 50 may attenuate the radiation and the fields generated during this examination, namely the magnetic fields used in MRI imaging.

FIGS. 2 and 3 show perspective views of the front and rear of the detection device. A set of foam wedge cushions 58 is provided on which the patient rests his or her head. The set of foam wedge cushions 58 comprises a lower portion extended by two lateral portions matching the shape of the detection device's shell 50. Two lateral portions press against the portions of the shell 50 that form the branches of the U-shape.

The detection device comprises a measuring device which will be described in greater detail later. An inlet connector 53 for the measuring device protrudes from the top of the shell 50, the other elements constituting the measuring device being arranged in the shell 50. The shell 50 also encloses an electronic management module.

The inhalator 51 is linked to the inlet connector via a linking tube 52. The inhalator is presented, for example, in the form of a mask covering the nose and mouth and comprising an anti-bacterial filter through which the patient breathes. The mask is held on the patient's head by an elastic band.

The patient thus breathes via the inhalator linked to the linking tube 52 and the measuring device opening into the open air. The space 54 in the open air by which the patient breathes is namely shown in FIG. 3. The connector 53 and measuring device are offset with respect to the area under analysis so as to remain outside this area but close to the breathing source.

The portability of the detection device thus enables the measuring device, through which the patient breathes, to be positioned as closely to the patient as possible. Thus, the air circuit via which the patient breathes is of a short length. Positioning the measuring device laterally with respect to the patient's head further enables the air circuit via which the patient breathes to be reduced. The reduced length of the circuit makes it possible to have a volume of air that is not entirely renewed but which is tolerable for the patient who breathes through this air circuit for the full duration of the examination.

The lower tether strap 57 passes through the buckles attached under the shell.

FIG. 4 shows a detection device 1 for the position of at least one moving area, analysed by medical imaging, of a patient's body where the external shell 50 is shown transparently.

The shell 50 is attached to a base plate 49 to form a housing case. Openings are arranged in the case and namely an opening 55 to expel hot air via the vent duct 32, openings 56 for the intake of air into the inside of the case and an opening into the open air of a space 54 through which the patient breathes.

The support plate 49 is U-shaped, the patient placing his or her head between the branches of the U. The shell 50 extends above the support plate 49. A tether strap 57 for the detection device 1 is attached to the edge of the support plate 49 and passes under the lower face of the support plate 49. The strap 57 makes it possible to attach the device to a mobile medical table, for example. The detection device is advantageously portable.

The case houses the measuring device whose inlet connector 53 protrudes with respect to the shell 50 of the case. A tube may thus be connected by which the patient breathes. The outlet connector 13 communicates with the space 54 in the open air, by which the patient breathes.

The measuring device is attached to a base 31 which is attached to the support plate 49. Removing the shell 50 provides access to the measuring device, which may then be removed, namely for sterilization.

The pressure propagation ducts 33 and 34 are fully housed in the case, as is the hot air supply duct 38. The air intake opening 48 is arranged inside the case. When the hot air is directed towards the central body of the measuring device, the air outside the shell penetrates into the shell via aeration openings 56 and is then drawn into the air intake opening 48. The air is made to move namely be the ventilator 43 activated in the hot air supply duct 38. The air is heated by the resistance 46 and regulated by the temperature sensor 47.

After having circulated around the central body, the hot air is evacuated, by the vent duct 32, outside the case.

The case comprises an electronic management module 30 arranged so as to supply data representative of the differential pressure measurement made between the upstream pressure and the downstream pressure, this data being processed to generate a digital data signal 40 representative of a respiratory volume of the patient in line with an incoming 41 or outgoing 42 digital synchronization signal.

The management module 30 comprises, for example, at least one printed circuit. The management module 30 comprises, for example, a data bus, an address bus and a control bus linking together the processing components, the memorization components and the interface components. The memory components are, for example, volatile or non-volatile memories. The processing components are, for example, of the FPGA (Field Programmable Gate Array), DSP (Digital Signal Processor) or ASIC (Application Specific Integrated Circuit) type. The electrical signals are, for example, of the TTL or CMOS type. Shall be designated by module, such as the management module or heating module, a functional assembly comprising a programme or sub-programme memorized and performed to process data or produce data and able to use a working memory space.

The detection device 1 is linked to an electric power cable 19.

The detection device 1 is linked to a communication link supplying an outgoing digital synchronization signal 42. This synchronization signal 42 is produced by the management module 30 using the data representative of the measured flow rate of the air flow.

The detection device 1 is linked to a communication link receiving an incoming digital synchronization signal 41.

The detection device 1 is linked to a communication link and supplies, on this line, an outgoing digital signal 40 representative of the respiratory volume of the patient, in line with a synchronization signal. This synchronization signal is the incoming or outgoing synchronization signal.

For the generation of this signal 40, the management module generates data representative of the respiratory volume of the patient using data representative of the measure flow rate of the air flow.

One example of the processing of the data produced, namely by the differential pressure sensor 37, will be described later with reference to FIG. 8.

FIG. 5 shows a perspective view of the measuring device to which a duct 38 supplying air at a regulated temperature, a vent duct 32 and pressure propagation ducts 33 and 34 are connected.

The exterior of the casing delimits a rectangle parallelepiped comprising longitudinal chamfers. The upper face comprises an opening to access a warming space and is linked to an attachment plate 14 for the linking duct 23. The warming space will be described in greater detail later.

The lower face comprises an opening to access to the warming space and is linked to the attachment plate 20 for the vent duct 32.

The front face comprises radial passages 28 opening opposite the upstream 10 and downstream 11 pressure measuring spaces. Connectors 29 are provided to be inserted into these radial passages 28 and attached to this front face. These connectors 29 are of a shape intended for the connection of two pressure propagation ducts 33 and 34 leading to the differential pressure measuring sensor 37.

The rear face is fitted, for example, with threaded holes for the attachment of a support stand for the measuring device.

The ring-shaped spaces 10 and 11 for measuring the differential pressures have been shown in dotted lines. The connectors 29 are arranged in the passages 28 communicating with spaces 10 and 11 are connected to a downstream pressure propagation duct 33 and to an upstream pressure propagation duct 34. The two pressure propagation ducts 33 and 34 are linked to a differential pressure sensor 37 that is offset with respect to the measuring device 3.

The pressure propagation ducts 33 and 34 are, for example, of a length of a few centimetres to a few tens of centimetres. The differential pressure sensor 37 closes each of the pressure propagation ducts 33 and 34 and comprises equipment to supply data representative of the differential pressure. The differential pressure sensor 37 thus supplies data representative of the difference in pressure between the upstream pressure and the downstream pressure in the measuring device. This data is given, for example, in the form of an analogue voltage or in the form of encoded digital data.

To warm the central body, a system of pulsed hot air enables the warming of the measuring device, the air then being evacuation via a vent duct 32.

The hot pulsed air system, offset with respect to the measuring device, comprises an electrical air heating resistance 46 arranged in a hot air supply duct 38. The heating resistance 46 is powered by a heating module 45. This heating module 45 may be controlled by a management module.

The hot air supply duct 38 is, for example, made of a material that is not electrically conductive so as to avoid any risk of current leakage. This duct 38 is connected to the linking duct 23 communicating with the warming space. The air penetrates into the hot air supply duct 38 by an air intake 48. Advantageously, no current circulates in the vicinity of the duct through which the patient breathes and in which the respiratory flow is to be measured.

The air entering by the air intake 48 is driven by a ventilator 43 set into movement by an actuator 39. The actuator 39 may itself be controlled by a management module or can be started as soon as the detection device in which the measuring device is installed is switched on.

A temperature sensor 47 is arranged in the hot air supply duct 38 and is linked to a temperature regulation module 44. This regulation module 44, for example, supplies the management module with data representative of the temperature of the air directed towards the measuring device. Regulating the heating air temperature thereby makes it possible to avoid the overheating of the measuring device thereby avoiding overheating the air inhaled by the patient.

The management module controls, for example, the heating module 45 to turn off the heating when the regulation module 44 supplies data representative of the exceeding of a safety threshold temperature stored in the memory of the management module.

Turning off the heating may also be controlled by a bimetallic strip acting as a temperature sensor for the air temperature and mounted in series in the electrical power supply circuit of the electrical heating resistance. A bimetallic strip may also be provided that is attached to one face of the external casing of the measuring device or in the heating air vent duct. The short-circuit or open-circuit state of the bimetallic strip can also be controlled by the management module.

After the heated air has circulated in the measuring device to warm it, the heating air is evacuated via a vent duct 32. The vent duct 32 namely enables the heating air to be directed out of an external protective shell.

FIG. 6 shows a longitudinal section of a measuring device 3 generating a differential pressure representative of the flow rate of a gaseous flow. This measuring device 3 comprises an inlet 5 and an outlet 6 for the gaseous flow whose flow rate is to be measured.

The terms used to designate the inlet 5 and outlet 6 for the gaseous flow are not restrictive. Shall be similarly designated the measurements made upstream or respectively downstream performed close to the inlet or respectively outlet. When the patient exhales, the gaseous flow enters by inlet 5 and exits by outlet 6, the flow circulates upstream to downstream.

On the contrary, when the patient inhales, the direction of the gaseous flow is reversed and enters by the outlet before passing via the inlet 5.

The inlet 5 of the gaseous flow is arranged towards the patient and the outlet 6 of the gaseous flow is arranged in a space in the open air. The shapes of the inlet 5 and outlet 6 vents are symmetrical and are tapered and of a length calculated to obtain the same flow rate measurement whether incoming or outgoing.

The measuring device 3 comprises a central body 8 surrounded by a casing 9. The ends of the body protrude at either end of the casing. A hollow connector 53 at the inlet 5 and a hollow connector at the outlet 6 are attached to the ends of the hollow body 8. The hollow body 8 comprises longitudinal channels 4 communicating first with the inlet 5 of the gaseous flow and secondly with the outlet 6 of the gaseous flow. Seals 15 are arranged between the central body 8 and the connectors 53 and 13 at the inlet and outlet. The connectors 13 and 53 are fitted onto the body 8.

Seals 7a, 7b, 7c and 7d arranged between the casing 9 and the central body 8 delimit a first space 10 to measure an upstream pressure and a second space 11 to measure a downstream pressure. The seals 7b and 7c arranged between the casing 9 and the central body 8 also delimit a third space 12 for warming the central body 8, this third space 12 being supplied with fluid at a regulated temperature. This fluid is, for example, air which is heated as described previously.

The seals are, for example, O-rings. Any type of ring-shaped seal may be selected, which is to say those with non-circular sections, such as quad-rings.

Four seals 7a, 7b, 7c and 7d successively delimit between each other, the first space 10, the third space 12 and the second space 11.

The casing 9 comprises an inner housing in which the central body 8 is positioned, this inner housing forms several bearings against which a seal is positioned to make an air-tight contact. The successive bearings made in the casing 9 are made with decreasing diameters going from one end of the casing abutting against a protruding peripheral shoulder 26 on the central body 8 to the other end of the casing 9 by which the central body 8 protrudes. The insertion of the central body 8 fitted with its seals is thereby facilitated.

Housing for seals 7a, 7b, 7c and 7d take the form of external peripheral grooves. The central body 8 also comprises housings, in the form of external peripheral grooves, delimiting the pressure measuring spaces. External peripheral grooves made in the central body 8 further delimit the cooling fins 25. The cooling fins 25 are in the warming space 12.

The central body 8 and the casing 9 fit into one another along their longitudinal axis. The casing 9 is then screwed to a shoulder 26 on the central body 8.

Seals 15 are arranged in housings made in the end collars on which the inlet and outlet connectors 13 and 53 are fitted.

The different elements constituting the measuring device can thus be disassembled, namely to be sterilized. In particular, the central body 8 and the inlet and outlet connectors 13 and 53 can be sterilized. The seals can be sterilized or replaced.

The casing 9 surrounding the central body 8 forms two points of access to the space 12 for warming up the central body 8. Plates 14 and 20 attached to the casing 9 comprise an opening in which a duct may be immobilised. These plates 14 and 20 are attached to the casing by screws.

The linking duct 23 is intended to be supplied with fluid at a controlled temperature. In FIG. 6 only the linking duct 23 is attached to the casing 9 by means of plate 14, the access in the other plate 20 being left free, but a vent duct linked to this other plate 20 has previously been described with reference to FIG. 4.

Heating-conducting fins 25 are arranged in the central body 8 and protrude into the warming space 12.

As shown in FIG. 7, these fins 25 are in the form of parallel crowns which delimit, between each other, peripheral grooves in the central body 8.

Warmed air is, for example, injected into the linking duct 23 and then passes through the opening 21 made in the casing to reach the warming space 12. The warm air thus warms the central body 8. The fins 25 enable a better distribution of the heat in the central body 8. The warming air injected into the warming space 12 then exits via the opening 22 made in the casing 9. This evacuated warm air is channeled into a vent duct as described previously. A vent duct is thus attached, in the opening in attachment plate 20 and in communication with the third warming space 12.

Warming the central body 8 makes it possible to avoid the condensation of the air exhaled by the patient which circulates in the central body 8.

The central body 8 comprises a network of parallel channels 4. These longitudinal channels 4 are spaced over the full diameter of the passage for the air flow arranged in the central body.

The air flow passing through these longitudinal channels 4 creates pressure in the longitudinal channels.

Radial ducts 17 and 18 are made in the central body 8 to link one or several longitudinal channels with spaces 10 and 11 to measure the pressures upstream and downstream.

Radial ducts 17 link the external longitudinal channels 4 with the space 11 for measuring the downstream pressure. Radial ducts 18 link the external longitudinal channels 4 with the space 10 for measuring the upstream pressure.

As spaces 10 and 11 for measuring the pressure are closed, the measurement of their internal pressure corresponds to that upstream and downstream in the longitudinal channels. These pressure measurements may thus be used to measure the flow rate of the air flow.

Spaces 10 and 11 for measuring the upstream and downstream pressure are delimited by the central body 8 and the casing 9 and, as described previously, ducts linked to these spaces 10 and 11 enable the propagation of their internal pressure. As described previously, a differential pressure sensor linked to these first and second pressure measuring spaces 10 and 11, make it possible for data representative of the differential pressure to be generated.

Warming the central body 8 as previously described makes it possible to avoid the condensation of the air and the appearance of water droplets which could block the longitudinal channels 4 or the radial ducts 17 and 18 thereby adversely affecting the pressure measurements.

FIG. 8 schematically shows an example of the organisation of the management module 30.

The management module 30 comprises a differential pressure sensor 37 linked to pressure propagation ducts 33 and 34. The differential pressure sensor 37 supplies data representative of the measured differential pressure read by an arithmetic calculation module 116 that supplies data representative of the measured flow rate. The arithmetic calculation module 116 performs, for example, a multiplication of the data representative of a differential pressure to calculate the data representative of a flow rate. The data representative of the measured flow rate are stored in a memory storage space 112.

The memory storage space 112 of the data representative of the measured flow rate is read by an outgoing synchronization signal generation module 113. This module 113, for example, performs comparisons between the successive values and determines the maximums and minimums of measured flow rate corresponding to synchronization fronts stored in a memory storage space 114 for the outgoing synchronization signal.

The memory storage space 114 for the outgoing synchronization signal is namely read by an interface 105 supplying the outgoing synchronization signal 42.

The memory storage space 112 for the data representative of the measured flow rate is read by a parameterizing module 111 for a respiration model. This parameterizing module 111 can access a memory storage space 110 for a non-parameterized respiration model. The respiration model corresponds to a curve representative of a volume of inhaled and exhaled air by a human being. The non-parameterized model 110 must therefore be parameterized according to each examination. The parameterizing module 111 of the respiration model thus provides access to the data 110 representative of the non-parameterized respiration model and to the data 112 representative of the measured flow rate to generate data 109 representative of the parameterized respiration model, this data being stored in a memory space 109.

The parameterizing module 111 of the respiration model performs an adjustment over a pre-determined number of respiratory cycles. A delay of a few tens of seconds is, for example, planned for the parameterizing of the respiration model. A delay of a few minutes can be planned during which the patient should fall into a regular breathing rhythm.

To adjust the parameterized respiration model, the parameterizing module 111 namely comprises a sub-programme to adjust the model's parameters.

Other sub-programmes may be provided in order to parameterize the model such as a self-learning programme to make successive adjustments and error assessments between each adjustment.

The respiration model is a model called a LUJAN model, expressed as:

Z(t)=Zo−B·(Cos(π·t/τ−φ))^{2N}

In this function, the position in metres of an organ is given by Z(t).

Zo is an adjustable parameter corresponding to the exhalation position.

B is an adjustable parameter corresponding to the depth of each breath.

Cos is the mathematical function, cosine.

π is the constant of a value of approximately 3.14.

t is the time variable expressed in seconds.

τ is an adjustable parameter corresponding to the period of the respiratory cycle.

φ is an adjustable parameter corresponding to a phase shift.

N is an adjustable parameter corresponding to a degree of asymmetry of the model.

These adjustable parameters are, for example, determined by several samplings and one or several solutions of equation systems.

Determination by equation systems may be combined with self-learning sub-programmes or mean value calculation programmes.

Other respiration models may thus be used.

After the memorizing of the parameterized respiration model 109, a module 115 to generate data representative of the respiratory volume performs a memory access to the parameterized respiration model 109 and to the data 112 representative of the respiration rate. This module 115 generates and writes the data representative of the patient's respiratory volume in a memory space 118.

For the generation of data 118 representative of the respiratory volume, the module 115 which generates it namely comprises a sub-programme for the digital integration of the flow rate.

The management module 30 comprises an interface 103 to receive an incoming synchronization signal 41. The data representative of the incoming synchronization signal is written, by this interface 103, into a memory storage space 108.

The management module 30 comprises an interface 102 to receive at least one command signal 101 for the selection of synchronization with an incoming signal or with an outgoing signal. Other commands may be received to pilot the management module 30. The data representative for this selection command is written, by this interface 102, in a memory storage space 107.

The management module 30 comprises a module 119 to generate the data representative of the patient's respiratory volume in line with an incoming or outgoing synchronization signal, this data being stored in a memory space 106. This memory space 106 is read by an interface 104 generating the outgoing transmission signal 40 for the data representative of the respiratory volume in line with the incoming or outgoing synchronization signal.

Module 119 namely provides access to the data 118 representative of the respiratory volume and to the incoming synchronization data 108 or to the outgoing synchronization data 114 to generate the respiratory volume data 106 in line with the incoming or outgoing synchronization signal. This generation module 119 namely comprises a data concatenation sub-programme. The combination of the respiratory volume data 118 with the incoming synchronization data 104 or with the outgoing synchronization data 114 is made as a function of the state of the memory space 107, accessed by the module 119 to generate the respiratory volume data 106 in line with the incoming or outgoing synchronization signal. The memory space 107 is put into a pre-determined state corresponding to the incoming or outgoing synchronization signal used.

The response time to process a variation of differential pressure translated into data representative of a variation in synchronized respiratory volume with one of the synchronization signals is, for example, less than 12 ms, which can correspond to the normal sampling frequency for a pre-determined pressure differential sensor. The differential pressure sensor is selected according to need. Thus, the management module may be organised so as to have this response time of 15 ms or 30 ms. A real-time system is thus obtained.

The generation of data representative of an activation authorization for the module 119 to generate the respiratory volume data 106 in line with the incoming or outgoing synchronization signal may also be provided. Such an authorization is, for example, generated by a module 117 to manage the operating temperature.

The module 117 to manage the operating temperature provides read and write access to the working memory spaces of the temperature regulation module 44, the heating module 45 and a control module 67 for the ventilator actuator 39.

The temperature regulation module 117 comprises, for example, a delay sub-programme according to a heating time of the measuring device and a sub-programme to control the heating to a memorized target temperature according to a measured temperature. The module 119 to generate the synchronized respiratory volume data accesses, for example, an authorization memory space in the temperature management module 117.

As shown in FIG. 9, the detection device 1 attached to the medical table 59 is moved into the medical imaging device 35 at the same time as the patient 2. The space formed by the shell 50 and foam wedge cushions 58 will be made sufficient for the patient to be able to position his or her head and hands. The positioning of the patient with his or her arms raised and hands locked behind his or her head allows better visualization of the area 60 to be analysed. The U-shape of the detection device in no way hinders the medical imaging process. The inlet connector 53 is namely offset with respect to the patient's head and to the area 60 of the patient to be analysed by medical imaging.

FIG. 10 shows medical imaging apparatus 35 comprising two medical imaging devices and detection device 1 for the position of the moving area, being analysed by medical imaging, of the patient's body 2.

Each imaging device comprises a stimulation and detection device 61 or 120, schematised by a ring 61 or 120, linked to an acquisition and control case 62 or 121 for the data representative of medical images. The medical image data 64 or 122 is transmitted by a communication link to a processing station 140 or 143. A storage space 141 or 144 is provided for this data which will be analysed later.

The signals transmitted by each medical imaging device and received by the processing station 140 or 143 correspond to data representative of medical images in line with the synchronization signal 123 supplied by the detection device or the synchronization signal 145 transmitted to it.

The communication links between the different stations or devices are coupled by an optical interface enabling an electrical insulation.

The medical imaging devices are, for example, of the type positron emission tomography (PET), computed tomography acquisition (CT) or magnetic resonance imaging (MRI).

Each medical imaging device is linked to the detection device 1 by which a synchronization signal 123 or 145 is transmitted.

This synchronization signal 145 is an incoming synchronization signal for the detection device 1 emitted, for example, by a computed tomography acquisition device.

The synchronization signal 123 is an outgoing synchronization signal for the detection device 1, received, for example, by a positron emission tomography device.

The detection device 1 is linked by its power cable 19 to a power supply unit 66. This power supply unit is connected to the power grid via an isolation transformer 124.

The communication or power supply cables linked to the detection device 1 are selected of a sufficient length to enable the medical table to translate inside the medical imaging device.

The detection device 1 is also linked to its processing station 65 to which it transmits data 40 representative of the respiratory volume in line with the incoming or outgoing synchronization signal. A storage space 142 is provided for this data which will be analysed later.

The processing stations 65, 140 or 143 are, for example, computers equipped with processing programmes and comprising user interfaces. The user interface comprises a screen and a keyboard. The processing stations 65, 140 and 143 are powered by the grid via an isolation transformer 124.

It is to be noted that systems 140 and 143 may be physically accommodated in a single system, and integrated into a control console and include 2D, 3D (and 4D with the time component incorporated by SPI into 3D structures) image reconstruction software.

The system thus formed is often called a reconstruction console.

There may be reconstruction consoles with only image processing software that are located in rooms at a greater or lesser distance from the examination area and connected by computer network.

FIGS. 11 and 12 show a calibration tool 68 for the detection device 1. A chamber 127 is delimited by a piston 126 controlled in translation. Another type of mobile wall delimiting the volume of the chamber 127 and connected to the target 125 may be used instead of the piston.

The calibration tool 68 comprises an aperture 128 supplied by the chamber 127 and connected to the measuring device. The chamber 127 is linked, by a linking tube 69, to the inlet connector 53 of the detection device 1. By controlling the movement of the piston 126 according to pre-determined cycles producing pre-determined air flows, the detection device 1 can be calibrated.

The chamber 127 is delimited by the mobile piston 126, which is also linked to a target 125. This target 125 can be detected by both medical imaging devices.

The target 125 is attached to a non-metallic rod 131, itself attached to an actuating head 132 of the piston 126.

The target 125 and mobile piston 126 are thus joined for their simultaneous displacement control.

One detail in FIG. 11 shows the actuator 129 of the piston. The actuator 129 comprises a control interface 130 to receive signals to command the forward or backward movement of the piston.

The rod 131 is attached to the target 125 by threading 136 made on the end of the rod 131. This threaded end is screwed into a threaded hole in a weight 133 made of a plastic material. This weight 133, for example spherical, comprises an internal housing 134 closed by a plug 135. A radioactive liquid may be inserted into the housing in the target 125. The radioactive liquid makes it possible for the target to be detected by a medical imaging device of the type positron emission tomography (PET). The material of the target makes it able to be detected by a medical imaging device of the type CT scan.

The calibration tool may be introduced with the detection device 1 into a medical imaging device. The detection device 1 may thus be calibrated at the same time as one of the medical imaging devices.

The calibration tool is advantageously used to synchronize the timers of one of the imaging devices and of the detection device. It is thus possible for the timers of the medical imaging devices to be synchronized with respect to one another. Indeed, although the electronic timers used are of great accuracy, they may be slightly out of synch thereby leading to inaccuracies in the measurements during the subsequent analysis of the data supplied by the different imaging devices.

The calibration tool may also be used in the case of a new measuring device being installed or when software in the detection device is updated or when the processing parameters are adjusted. An inspection may also be performed by way of precaution.

It must be obvious for one skilled in the art that the present invention enables other variant embodiments. Consequently, the present embodiments must be considered as merely illustrative of the invention defined by the attached claims.

1. A device to detect the position of at least one moving part of the body of a patient analysed by medical imaging, wherein it comprises a management module comprising electronic and electric components including at least one differential pressure sensor linked to a measuring device generating a representative differential pressure of the patient's respiratory flow rate, the management module being organised to perform at least:
the acquisition and processing of data produced by the differential pressure sensor for the generation of data representative of the respiratory volume and for the generation of an outgoing digital synchronization signal,
the reception of an incoming digital synchronization signal,
the generation of an outgoing digital signal of data representative of the respiratory volume in line with one of the two incoming or outgoing digital synchronization signals.

2. A detection device according to claim 1, wherein the outgoing digital synchronization signal is representative of the detection of extremes with respect to the respiratory movement corresponding to the minimum or maximum respiration flow rate of a defined portion of the respiratory cycle.

3. A detection device according to claim 1, wherein the management module is adapted for the reception of a selection command for one of the two incoming or outgoing digital synchronization signals for the generation of the outgoing digital signal of the data representative of the respiratory volume in line with one of the two incoming or outgoing digital synchronization signals.

4. A detection device according to claim 1, wherein the management module is organized so as to generate the digital signal of the data representative of the respiratory volume in line with the incoming or outgoing digital synchronization signal, with a response time of less than or equal to 30 ms or 15 ms or even 12 ms with respect to the variation in data produced by the differential pressure sensor.

5. A detection device according to claim 1, wherein the data representative of the respiratory volume is calculated from a memorized parameterizable model corresponding to a curve representing the volume of air exhaled and inhaled by a patient, the data representative of the patient's respiratory flow rate being processed by a parameterizing module of the model over a pre-determined number of respiratory cycles for a memorization of data representative of the parameterized model, then being processed by a real-time adjustment module of the parameterized model for the generation of data representative of the respiratory volume.

6. A detection device according to claim 1, wherein it comprises a portable case enclosing at least the management module and offset with respect to the moving part being analysed.

7. A detection device according to claim 6, wherein it comprises an inhalator through which the patient breathes, this inhalator being linked to a tube connected at the inlet of the measuring device, the measuring device being attached to the case.

8. Medical imaging apparatus comprising at least one medical imaging device synchronized with a device to detect the position of the moving part of the patient's body being analysed by medical imaging according to claim 1.

9. Medical imaging apparatus according to claim 8, wherein it comprises at least two medical imaging devices each of a different type and being of the type positron emission tomography (PET), computed tomography acquisition (CT) or magnetic resonance imaging (MRI).

10. Medical imaging apparatus according to claim 9, wherein it comprises a calibration tool for the detection device and said medical imaging devices, the calibration tool comprising an aperture supplied by a chamber and connected to the measuring device, the chamber being delimited by a mobile wall linked to a target able to be detected by said medical imaging devices, the target and mobile wall being joined for the simultaneous control of their displacement, the calibration tool ensuring a synchronization of the timers and medical imaging devices.
